(19) United States
(12) Patent Application Publication
(10) Pub. No.: US 2020/0143657 A1
(43) Pub. Date: May 7, 2020

(54) Medical Alert System

(71) Applicant: Patrick Humphrey, Cumming, GA (US)

(72) Inventor: Patrick Humphrey, Cumming, GA (US)

(21) Appl. No.: 16/182,722

(22) Filed: Nov. 7, 2018

(51) Int. Cl. G08B 21/04; G16H 40/67; G16H 80/00

(52) U.S. Cl. CPC G08B 21/0453; G16H 80/00; G16H 40/67

(57) ABSTRACT

A medical alert system for actively monitoring biological parameters of a user includes a computer system and an electronics module. The computer system is coupled to a computer network. The electronics module is coupled to a band that is configured to couple around a wrist of a user. The electronics module comprises a plurality of first sensors and a transceiver. Each first sensor is configured to assess a respective biological parameter of the user. The electronics module is configured to selectively and wirelessly communicate the respective biological parameter to the computer system and a primary care physician of the user, positioning an operator of the computer system and the primary care physician to selectively contact emergency personnel to assist the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to alert systems and more particularly pertains to a new alert system for actively monitoring biological parameters of a user.

### BRIEF SUMMARY OF THE INVENTION

An embodiment of the disclosure meets the needs presented above by generally comprising a computer system and an electronics module. The computer system is coupled to a computer network. The electronics module is coupled to a band that is configured to couple around a wrist of a user. The electronics module comprises a plurality of first sensors and a transceiver. Each first sensor is configured to assess a respective biological parameter of the user. The electronics module is configured to selectively and wirelessly communicate the respective biological parameter to the computer system and a primary care physician of the user, positioning an operator of the computer system and the primary care physician to selectively contact emergency personnel to assist the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an in-use view of a medical alert system according to an embodiment of the disclosure.

FIG. 2 is a top view of an embodiment of the disclosure.

FIG. 3 is a bottom view of an embodiment of the disclosure.

FIG. 4 is an isometric perspective view of an embodiment of the disclosure.

FIG. 5 is a side view of an embodiment of the disclosure.

FIG. 6 is a flow diagram of an embodiment of the disclosure.

FIG. 7 is a block diagram view of an embodiment of the disclosure.

### DETAILED DESCRIPTION OF THE INVENTION

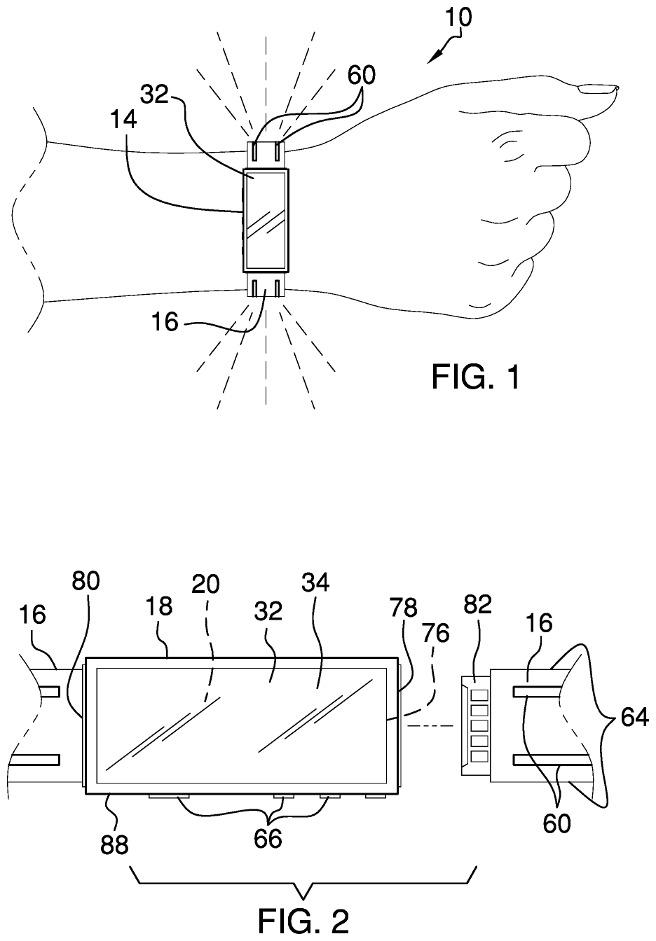

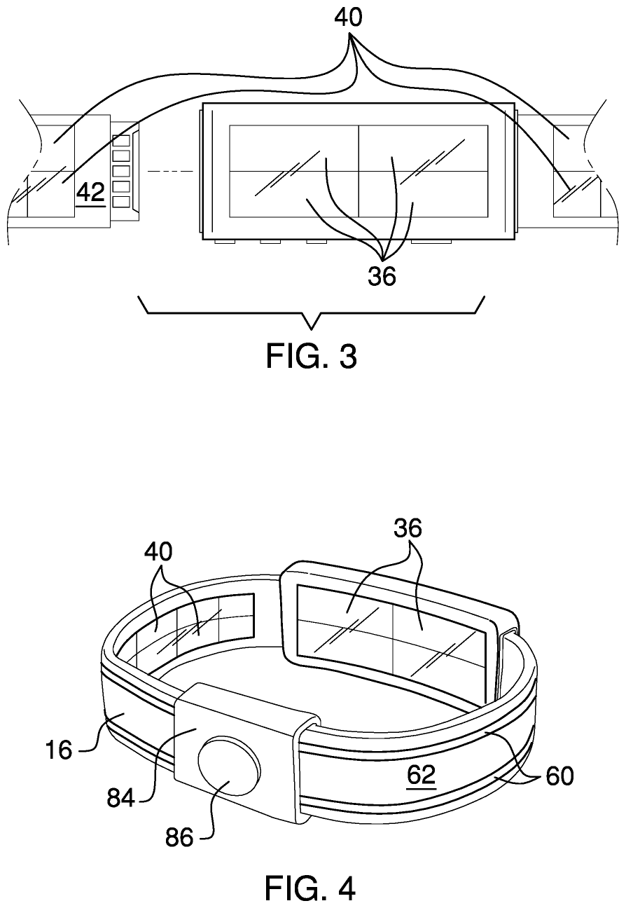

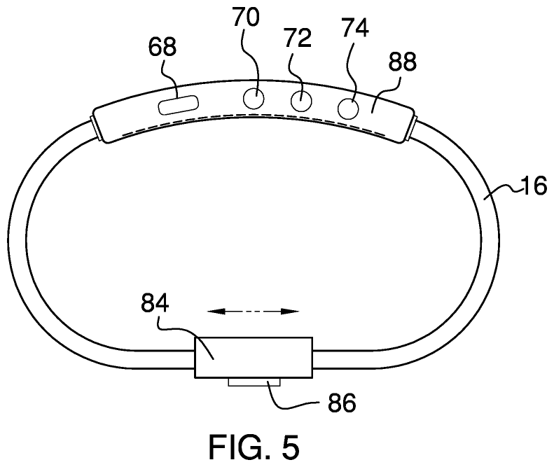

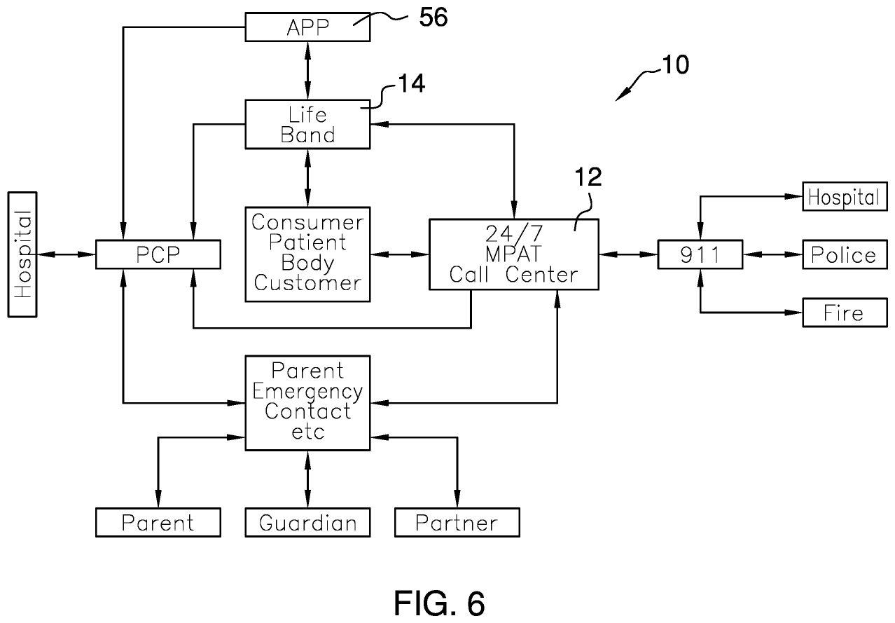

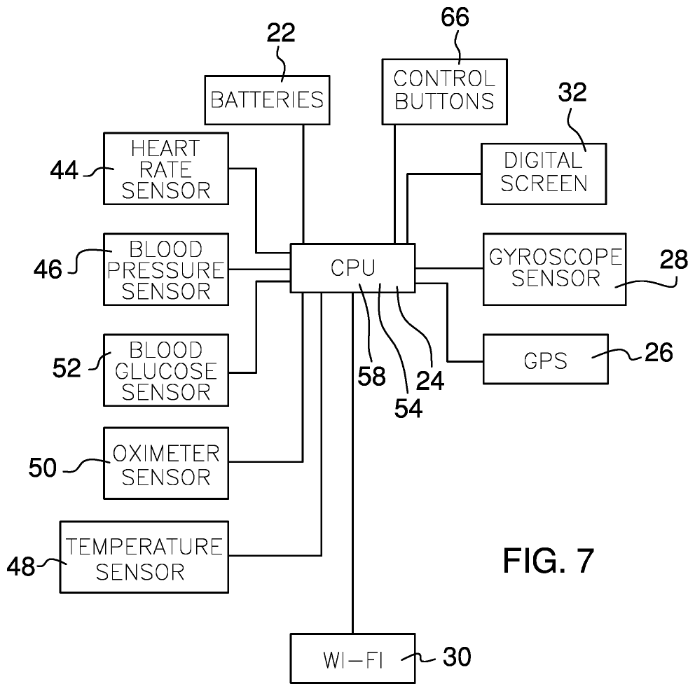

With reference now to the drawings, and in particular to FIGS. 1 through 7 thereof, a new alert system embodying the principles and concepts of an embodiment of the disclosure and generally designated by the reference numeral 10 will be described.

As best illustrated in FIGS. 1 through 7, the medical alert system 10 generally comprises a computer system 12 and an electronics module 14. The computer system 12 is coupled to a computer network and would be a component of a 24 hour, 7 days per week call center. The electronics module 14 is coupled to a band 16 that is configured to couple around a wrist of a user.

The electronics module 14 comprises a housing 18 that defines an internal space 20. A battery 22, a microprocessor 24, a receiver 26, a gyroscope 28, and a transceiver 30 are coupled to the housing 18 and are positioned in the internal space 20. A screen 32 is coupled to a top 34 of the housing 18. The microprocessor 24 is operationally coupled to the battery 22, the receiver 26, the gyroscope 28, the transceiver 30, and the screen 32.

The transceiver 30 is configured for wireless local area networking and can communicate with the computer system 12 at the call center. The receiver 26 is global positioning system enabled and is configured to receive location coordinates of the user, positioning the receiver 26 to relay the location coordinates to the microprocessor 24. The gyroscope 28 is configured to detect a rapid positional displacement, as would occur in event of the user falling, and to communicate the positional displacement to the microprocessor 24. In the event of a fall, the call center would be apprised and an operator at the call center would be positioned to dispatch emergency personnel to the location coordinates of the user.

A plurality of first sensors 36 is coupled to a bottom 38 of the housing 18 so that the first sensors 36 are positioned in contact with the wrist of the user. A plurality of second sensors 40 is coupled to an inner face 42 of the band 16 so that the second sensors 40 are positioned in contact with the wrist of the user. The first sensors 36 and the second sensors 40 are operationally coupled to the microprocessor 24 and are dry-skin type. Each first sensor 36 and each second sensor 40 is configured to assess a respective biological parameter of the user utilizing techniques and components well known to those skilled in the art of assessment of biological parameters.

The plurality of first sensors 36 and the plurality of second sensors 40 comprise a heart rate/pulse monitor 44, a blood pressure sensor 46, a temperature sensor 48, a pulse oximeter 50, and a glucose meter 52. The heart rate/pulse monitor 44 is configured to measure a heart rate and a pulse of the user. The blood pressure sensor 46 is configured to measure a blood pressure of the user. The temperature sensor 48 is configured to measure a temperature of the user. The pulse oximeter 50 is configured to measure a blood oxygen content of the user. The glucose meter 52 is configured to measure a glucose level of the user.

The microprocessor 24 is positioned to compel the screen 32 to selectively display the respective biological parameter. The screen 32 is touch enabled so that the screen 32 is configured to be selectively touched to enter commands into the microprocessor 24.

The electronics module 14 is configured to wirelessly communicate with both the computer system 12 and a primary care physician of the user. The microprocessor 24 is positioned to compel the transceiver 30 to selectively and wirelessly communicate the respective biological parameter, the positional displacement, and the location coordinates of the user to the computer system 12 and the primary care physician. The operator of the computer system 12 and the primary care physician are positioned to selectively contact emergency personnel, such as a police department, a fire department, and a hospital, to assist the user. Additionally, the user may elect, in advance, to have the operator of the computer system 12 or the primary care physician contact a designated emergency contact, such as a parent, a guardian, or a partner.

Communications programming code 54 is positioned on the microprocessor 24 the enables the user to choose from a plurality of modes wherein the respective biological parameter, the positional displacement, and the location coordinates of the user are communicated selectively to at least one of the computer system 12 and the primary care physician of the user. The operator of the computer system 12 and the primary care physician are positioned to selectively contact emergency personnel to assist the user.

Secondary device programming code 56 is positioned on an electronic device of the user that enables the electronic device to receive the respective biological parameter, the positional displacement, and the location coordinates of the user from the transceiver 30 and to transmit the respective biological parameter, the positional displacement, and the location coordinates of the user to the primary care physician. The primary care physician then is positioned to selectively contact emergency personnel to assist the user. The secondary device programming code 56 also enables the primary care physician to selectively activate and to set parameters for each of the plurality of first sensors 36 and each of the plurality of second sensors 40.

Display programming code 58 is positioned on the microprocessor 24 that enables the microprocessor 24 to compel the screen 32 to scroll a message that is received from the primary care physician or the computer system 12. The message could include instructions such as actions the user could take to remedy an aberrant biological parameter or to seek medical attention.

A light strip 60 is coupled to an outer face 62 of the band 16. The light strip 60 is operationally coupled to the battery 22. The light strip 60 comprises a pair of light strips 60 that is positioned singly proximate to opposing edges 64 of the band 16.

A plurality of buttons 66, each of which is depressible, is coupled to a side 88 of the housing 18. The plurality of buttons 66 comprises a power button 68, a select button 70, and a reset button 72. The power button 68 is configured to be selectively depressed to couple the microprocessor 24 to the battery 22 to active the electronics module 14. The select button 70 is configured to be selectively depressed to select a respective first sensor 36 or a respective second sensor 40. The reset button 72 is configured to be selectively depressed to reset the respective first sensor 36 or the respective second sensor 40.

The plurality of buttons 66 also comprises a light button 74. The light button 74 is configured to be depressed to operationally couple the light strip 60 to the battery 22. The light strip 60 is configured to illuminate to facilitate locating of the user by emergency personnel.

A first connector 76 is coupled to a first endpoint 78 of the housing 18. The band 16 is coupled to and extends from a second endpoint 80 of the housing 18. A second connector 82 is coupled to the band 16 distal from the housing 18. The second connector 82 is complementary to the first connector 76. The second connector 82 is positioned to selectively couple to the first connector 76 to couple the band 16 around the wrist of the user.

An adjuster 84 is coupled to the band 16 substantially equally distant from the second endpoint 80 of the housing 18 and the second connector 82, as shown in FIG. 4. The adjuster 84 is configured to selectively couple to the band 16 so that the second connector 82 is selectively positionable relative to the second endpoint 80 of the housing 18, rendering the band 16 adjustable to a size of the wrist of the user.

A knob 86, which is depressible, is operationally coupled to the adjuster 84. The knob 86 is configured to be depressed to decouple the adjuster 84 from the band 16 so that the second connecter is selectively positionable relative to the second endpoint 80 of the housing 18. The knob 86 is configured to be released to fixedly position the second connecter relative to the second endpoint 80 of the housing 18.

In use, the band 16 is coupled around the wrist of the user, positioning the first sensors 36 and the second sensors 40 to assess their respective biological parameters. The user simply goes about their daily activities. Should the user fall or a respective biological parameter indicate that an emergency medical situation is evolving, the operator of the computer system 12 and the primary care physician are positioned to selectively contact emergency personnel.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A medical alert system comprising:
a computer system coupled to a computer network;
a band configured for coupling around a wrist of a user; and
an electronics module coupled to the band, the electronics module comprising:
a plurality of first sensors wherein each first sensor is configured for assessing a respective biological parameter of the user, and
a transceiver wherein the electronics module is configured for selectively wirelessly communicating the respective biological parameter of the user to the computer system and a primary care physician positioning an operator of the computer system and the primary care physician for selectively contacting emergency personnel to assist the user
a housing defining an internal space,
a battery coupled to the housing and positioned in the internal space,
a microprocessor coupled to the housing and positioned in the internal space, the microprocessor being operationally coupled to the battery,
the transceiver being coupled to the housing and positioned in the internal space, the transceiver being operationally coupled to the microprocessor, the transceiver being configured for wireless local area networking,
the first sensors being coupled to a bottom of the housing such that the first sensors are positioned in contact with the wrist of the user, the first sensors being operationally coupled to the microprocessor; and
a plurality of second sensors operationally coupled to the microprocessor, the second sensors being coupled to an inner face of the band such that the second sensors are positioned in contact with the wrist of the user wherein each second sensor is configured for assessing a respective biological parameter of the user;
a pair of light strips positioned singly proximate to opposing edges of the band, each light strip being operationally coupled to the battery;
a plurality of buttons coupled to a side of the housing, the buttons being depressible, the plurality of buttons comprising a power button, a select button, and a reset button wherein the power button is configured for selectively depressing for coupling the microprocessor to the battery, each of the select buttons is configured for selectively depressing for selecting a respective first sensor or a respective second sensor, and the reset button is configured for selectively depressing for resetting the respective first sensor or the respective second sensor, the plurality of buttons comprising a light button wherein the light button is configured for depressing for operationally coupling the light strip to the battery wherein the light strip is configured for illuminating for facilitating locating of the user by emergency personnel.

2. (canceled)

3. The system of claim 1, further including the first sensors and the second sensors being dry-skin type.

4. The system of claim 1, further including the plurality of first sensors and the plurality of second sensors comprising:
a heart rate/pulse monitor wherein the heart rate/pulse monitor is configured for measuring a heart rate and a pulse of the user;
a blood pressure sensor wherein the blood pressure sensor is configured for measuring a blood pressure of the user;
a temperature sensor wherein the temperature sensor is configured for measuring a temperature of the user;
a pulse oximeter wherein the pulse oximeter is configured for measuring a blood oxygen content of the user; and
a glucose meter wherein the glucose meter is configured for measuring a glucose level of the user.

5. The system of claim 1, further including a receiver coupled to the housing and positioned in the internal space, the receiver being operationally coupled to the microprocessor, the receiver being global positioning system enabled wherein the receiver is configured for receiving location coordinates of the user such that the receiver is positioned to relay the location coordinates to the microprocessor positioning the microprocessor to selectively compel the transceiver to wirelessly communicate the location coordinates of the user to the computer system and the primary care physician positioning the operator of the computer system and the primary care physician for selectively contacting emergency personnel to assist the user.

6. The system of claim 5, further including a gyroscope coupled to the housing and positioned in the internal space, the gyroscope being operationally coupled to the microprocessor wherein the gyroscope is configured for detecting a rapid positional displacement of the user and for communicating the displacement to the microprocessor positioning the microprocessor to selectively compel the transceiver to wirelessly communicate the rapid positional displacement of the user to the computer system and the primary care physician positioning the operator of the computer system and the primary care physician for selectively contacting emergency personnel to assist the user.

7. The system of claim 1, further including a screen coupled to a top the housing, the screen being operationally coupled to the microprocessor wherein the microprocessor is positioned to compel the screen to selectively display the respective biological parameter.

8. The system of claim 7, further including the screen being touch enabled wherein the screen is configured for selectively touching for entering commands into the microprocessor.

9. The system of claim 1, further comprising:
a first connector coupled to a first endpoint of the housing, the band being coupled to and extending from a second endpoint of the housing; and
a second connector coupled to the band distal from the housing, the second connector being complementary to the first connector wherein the second connector is positioned for selectively coupling to the first connector for coupling the band around the wrist of the user.

10. The system of claim 9, further including an adjuster coupled to the band substantially equally distant from the second endpoint of the housing and the second connector, the adjuster being configured for selective coupling to the band such that the second connector is selectively positionable relative to the second endpoint of the housing wherein the band is adjustable to a size of the wrist of the user.

11. The system of claim 10, further including a knob operationally coupled to the adjuster, the knob being depressible wherein the knob is configured for depressing for decoupling the adjuster from the band such that the second connecter is selectively positionable relative to the second endpoint of the housing and wherein the knob is configured for releasing for fixedly positioning the second connecter relative to the second endpoint of the housing.

12. The system of claim 6, further including communications programming code positioned on the microprocessor enabling the user for choosing from a plurality of modes wherein the respective biological parameter, the positional displacement, and the location coordinates of the user are communicated selectively to at least one of the computer system and the primary care physician of the user positioning the operator of the computer system and the primary care physician for selectively contacting emergency personnel to assist the user.

13. The system of claim 12, further including secondary device programming code positioned on an electronic device of the user enabling the electronic device for receiving the respective biological parameter, the positional displacement, and the location coordinates of the user from the transceiver, for transmitting the respective biological parameter, the positional displacement, and the location coordinates of the user to the primary care physician positioning the primary care physician for selectively contacting emergency personnel to assist the user, and for enabling the primary care physician for selectively activating and setting parameters for each of the plurality of first sensors and each of the plurality of second sensors.

14. The system of claim 13, further comprising:
a screen coupled to a top the housing; and
display programming code positioned on the microprocessor enabling the microprocessor for compelling the screen for scrolling a message received from the primary care physician or the computer system.

15. (canceled)

16. (canceled)

17. (canceled)

18. A medical alert system comprising:
a computer system coupled to a computer network;
a band configured for coupling around a wrist of a user;
an electronics module coupled to the band, the electronics module comprising:
a housing defining an internal space,
a battery coupled to the housing and positioned in the internal space,
a microprocessor coupled to the housing and positioned in the internal space, the microprocessor being operationally coupled to the battery,
a plurality of first sensors wherein each first sensor is configured for assessing a respective biological parameter of the user, the first sensors being dry-skin type, the first sensors being coupled to a bottom of the housing such that the first sensors are positioned in contact with the wrist of the user, the first sensors being operationally coupled to the microprocessor,
a receiver coupled to the housing and positioned in the internal space, the receiver being operationally coupled to the microprocessor, the receiver being global positioning system enabled wherein the receiver is configured for receiving location coordinates of the user such that the receiver is positioned to relay the location coordinates to the microprocessor,
a gyroscope coupled to the housing and positioned in the internal space, the gyroscope being operationally coupled to the microprocessor wherein the gyroscope is configured for detecting a rapid positional displacement of the user and for communicating the displacement to the microprocessor,
a transceiver wherein the electronics module is configured for wireless communication with the computer system and a primary care physician of the user such that the microprocessor is positioned to compel the transceiver to selectively wirelessly communicate the respective biological parameter, the positional displacement, and the location coordinates of the user to the computer system and the primary care physician positioning an operator of the computer system and the primary care physician for selectively contacting emergency personnel to assist the user, the transceiver being coupled to the housing and positioned in the internal space, the transceiver being operationally coupled to the microprocessor, the transceiver being configured for wireless local area networking, and
a screen coupled to a top the housing, the screen being operationally coupled to the microprocessor wherein the microprocessor is positioned to compel the screen to selectively display the respective biological parameter, the screen being touch enabled wherein the screen is configured for selectively touching for entering commands into the microprocessor;
a plurality of second sensors operationally coupled to the microprocessor, the second sensors being coupled to an inner face of the band such that the second sensors are positioned in contact with the wrist of the user wherein each second sensor is configured for assessing a respective biological parameter of the user, the second sensors being dry-skin type, the plurality of first sensors and the plurality of second sensors comprising:
a heart rate/pulse monitor wherein the heart rate/pulse monitor is configured for measuring a heart rate and a pulse of the user,
a blood pressure sensor wherein the blood pressure sensor is configured for measuring a blood pressure of the user,
a temperature sensor wherein the temperature sensor is configured for measuring a temperature of the user,
a pulse oximeter wherein the pulse oximeter is configured for measuring a blood oxygen content of the user, and
a glucose meter wherein the glucose meter is configured for measuring a glucose level of the user;
a first connector coupled to a first endpoint of the housing, the band being coupled to and extending from a second endpoint of the housing;
a second connector coupled to the band distal from the housing, the second connector being complementary to the first connector wherein the second connector is positioned for selectively coupling to the first connector for coupling the band around the wrist of the user;
an adjuster coupled to the band substantially equally distant from the second endpoint of the housing and the second connector, the adjuster being configured for selective coupling to the band such that the second connector is selectively positionable relative to the second endpoint of the housing wherein the band is adjustable to a size of the wrist of the user;
a knob operationally coupled to the adjuster, the knob being depressible wherein the knob is configured for depressing for decoupling the adjuster from the band such that the second connecter is selectively positionable relative to the second endpoint of the housing and wherein the knob is configured for releasing for fixedly positioning the second connecter relative to the second endpoint of the housing;
communications programming code positioned on the microprocessor enabling the user for choosing from a plurality of modes wherein the respective biological parameter, the positional displacement, and the location coordinates of the user are communicated selectively to at least one of the computer system and the primary care physician of the user positioning the operator of the computer system and the primary care physician for selectively contacting emergency personnel to assist the user;
secondary device programming code positioned on an electronic device of the user enabling the electronic device for receiving the respective biological parameter, the positional displacement, and the location coordinates of the user from the transceiver, for transmitting the respective biological parameter, the positional displacement, and the location coordinates of the user to the primary care physician positioning the primary care physician for selectively contacting emergency personnel to assist the user, and for enabling the primary care physician for selectively activating and setting parameters for each of the plurality of first sensors and each of the plurality of second sensors;
display programming code positioned on the microprocessor enabling the microprocessor for compelling the screen for scrolling a message received from the primary care physician or the computer system;
a pair of light strips positioned singly proximate to opposing edges of the band, each light strip being coupled to an outer face of the band, each light strip being operationally coupled to the battery; and
a plurality of buttons coupled to a side of the housing, the buttons being depressible, the plurality of buttons comprising a power button, a select button, and a reset button wherein the power button is configured for selectively depressing for coupling the microprocessor to the battery, the select button is configured for selectively depressing for selecting a respective first sensor or a respective second sensor, and the reset button is configured for selectively depressing for resetting the respective first sensor or the respective second sensor, the plurality of buttons comprising a light button wherein the light button is configured for depressing for operationally coupling the light strip to the battery wherein the light strip is configured for illuminating for facilitating locating of the user by emergency personnel.
